(19) United States
(12) Patent Application Publication
(10) Pub. No.: US 2025/0281209 A1
(43) Pub. Date: Sep. 11, 2025

(54) STERNAL CLOSURE SYSTEMS

(71) Applicant: ARTHREX, INC., Naples, FL (US)

(72) Inventors: Marcela MONTOYA, Cape Coral, FL (US); Derek SULLIVAN, Naples, FL (US); Steven ROLENC, Naples, FL (US); Andrew Christian PETRY, Naples, FL (US); Peter J. DREYFUSS, Naples, FL (US); Marc GERDISH, Zionsville, IN (US); Daniel T. ENGELMAN, Longmeadow, MA (US); Paul DIGIORGI, Fort Myers, FL (US); Patrick RUDERSDORF, Golden, CO (US)

(21) Appl. No.: 19/074,633

(22) Filed: Mar. 10, 2025

Related U.S. Application Data
(60) Provisional application No. 63/563,689, filed on Mar. 11, 2024.
(60) Provisional application No. 63/686,908, filed on Aug. 26, 2024.
(60) Provisional application No. 63/756,965, filed on Feb. 11, 2025.

(51) Int. Cl. A61B 17/80; A61B 17/84; A61B 17/88

(52) U.S. Cl. CPC A61B 17/8076; A61B 17/844; A61B 17/8872

(57) ABSTRACT

Sternal closure systems are provided for closing a separated sternum following a surgery that requires accessing a patient's chest cavity. An exemplary sternal closure system may utilize a combination of one or more bone plates, cerclages, and fixation devices.

### CROSS-REFERENCE TO RELATED APPLICATIONS

This application claims the benefit of U.S. Provisional Application No. 63/563,689, which was filed on Mar. 11, 2024.

The application further claims the benefit of U.S. Provisional Application No. 63/686,908, which was filed on Aug. 26, 2024.

This application further claims the benefit of U.S. Provisional Application No. 63/756,965, which was filed on Feb. 11, 2025.

The disclosures of each of the above applications are incorporated herein by reference in their entirety.

### BACKGROUND

This disclosure relates to the field of surgery, and more particularly to sternal closure systems for performing sternal closure procedures.

A sternotomy is a surgical procedure performed to gain access to a patient's chest cavity, such as when performing heart surgery, for example. The sternotomy typically requires separating the sternum into two parts. The separated sternum must be rejoined or “closed” after completing the surgery. Known sternal closure techniques involve the use of cerclage wires and/or a combination of bone plates and screws for reapproximating and rejoining the sternum bone sections that were separated during the surgery.

### SUMMARY

This disclosure relates to sternal closure systems for closing a separated sternum following a surgery that requires access to a patient's chest cavity. The sternal closure systems may utilize a combination of one or more bone plates, cerclages, and fixation devices.

An exemplary sternal closure system may include, inter alia, a bone plate including an opening, and a fixation device deployment assembly including a locking tab, a molly anchor, and a deployment pin. When the locking tab is engaged to the fixation device deployment assembly, the fixation device deployment assembly is configured to insert the molly anchor through the opening of the bone plate. When the locking tab is disengaged from the fixation device deployment assembly, the fixation device deployment assembly is configured to cause the deployment pin to move the molly anchor from a relaxed position to a deployed position.

An exemplary surgical method may include, inter alia, positioning a bone plate relative to a sternum, advancing a molly anchor of a fixation device deployment assembly through an opening of the bone plate and into the sternum while a locking tab is secured to the fixation device deployment assembly, removing the locking tab from the fixation device deployment assembly, and advancing a deployment pin to cause the molly anchor to transition from a relaxed position to a deployed position relative to the sternum.

An exemplary method may include, inter alia, advancing a molly anchor of a fixation device deployment assembly through an opening of a bone plate while a locking tab is secured to the fixation device deployment assembly, removing the locking tab from the fixation device deployment assembly, and advancing a deployment pin to cause the molly anchor to transition from a relaxed position to a deployed position relative to the bone plate.

Another exemplary sternal closure system may include, inter alia, a bone plate including a body having a first longitudinal section, a second longitudinal section, a bridging section extending between the first longitudinal section and the second longitudinal section, a bone contacting surface, and an outer surface opposite of the bone contacting surface. A ridge protrudes from the bone contacting surface and is configured to align and center the bone plate between a first sternum bone section and a second sternum bone section of a sternum.

Another exemplary sternal closure system may include, inter alia, a bone plate including a body having a first longitudinal section, a second longitudinal section, a bridging section extending between the first longitudinal section and the second longitudinal section, a bone contacting surface, and an outer surface opposite of the bone contacting surface. A first longitudinal slot is provided within the first longitudinal section, and a second longitudinal slot is provided within the second longitudinal section.

Another exemplary sternal closure system may include, inter alia, a bone plate including a through-passage, a locking ferrule received within the through-passage and including a one-way locking mechanism, and a cerclage including a suture received through the locking ferrule and locked relative to the bone plate by the one-way locking mechanism.

Another exemplary sternal closure system may include, inter alia, a bone plate including an opening, a screw received within the opening and including a head portion, a threaded portion, and a cross-pin mounted within the head portion, and a suture looped around the cross-pin and configured to secure a tissue relative to the bone plate.

Another example sternal closure system may include, inter alia, a bone plate including an opening, and a carrier configured to selectively attach to and detach from the bone plate. The carrier may be configured to temporarily hold a fixation device relative to the opening such that, when the carrier is attached to the bone plate, the fixation device vertically overlaps the opening. Further, upon application of force, the fixation device is configured to release from the carrier and at least partially pass through the opening to fix the bone plate relative to a sternum.

Another example sternal closure system may include, inter alia, a bone plate including an opening, and a fixation device configured to fix the bone plate relative to a sternum. The fixation device may be configurable between a relaxed position and a deployed position. Further, the fixation device may include first and second deployable legs on opposite sides of a slit. The first deployable leg may be longer than the second deployable leg.

Another example sternal closure system may include, inter alia, a bone plate including an opening, and a fixation device configured to fix the bone plate relative to a sternum. The fixation device may be configurable between a relaxed position and a deployed position. Further, the fixation device may include a molly anchor and a wedge anchor, and the wedge anchor may include an eyelet coupled to suture.

Yet another example sternal closure system may include, inter alia, a bone plate including an opening, and a pin inserter including at least one pin. Further, the at least one pin may be configured to be selectively advanced out of the pin inserter. The system may further include a fixation device configured to fix the bone plate relative to a sternum. Further, the fixation device may be configurable between a relaxed position and a deployed position. The fixation device may include a molly anchor, and the molly anchor may be moveable from the relaxed position to the deployed position as the at least one pin comes into contact with first and second deployable legs of the molly anchor.

The embodiments, examples, and alternatives of the preceding paragraphs, the claims, or the following description and drawings, including any of their various aspects or respective individual features, may be taken independently or in any combination. Features described in connection with one embodiment are applicable to all embodiments, unless such features are incompatible.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 schematically illustrates a sternum of a human thorax. A sternal closure system is utilized to reapproximate and rejoin sternum bone sections that were separated as a result of a sternotomy.

FIG. 2 is a top view of an exemplary bone plate of a sternal closure system.

FIG. 3 is a bottom view of the bone plate of FIG. 2.

FIG. 4 is an end view of the bone plate of FIG. 2.

FIG. 5 illustrates another exemplary bone plate of a sternal closure system.

FIG. 6 illustrates another exemplary bone plate of a sternal closure system.

FIG. 7 illustrates another exemplary bone plate of a sternal closure system.

FIG. 8 illustrates another exemplary bone plate of a sternal closure system.

FIG. 9 illustrates another exemplary bone plate of a sternal closure system.

FIG. 10 illustrates yet another exemplary bone plate of a sternal closure system.

FIG. 11 illustrates an exemplary fixation device of a sternal closure system.

FIG. 12 is a top view of the fixation device of FIG. 11.

FIG. 13 illustrates an initial position of deployable legs of a molly anchor of the fixation device of FIG. 11.

FIG. 14 illustrates a deployed position of deployable legs of the molly anchor of the fixation device of FIG. 11.

FIG. 15 illustrates another exemplary fixation device of a sternal closure system.

FIG. 16 schematically illustrates fixation of a bone plate to a bone using a plurality of fixation devices configured in the manner shown in FIG. 15.

FIG. 17 is a top view of another exemplary bone plate of a sternal closure system.

FIG. 18 is a side view of the bone plate of FIG. 17.

FIG. 19 illustrates an arrangement of locking barbs of a locking ferrule of the sternal closure system of FIGS. 17-18.

FIG. 20 illustrates an example assembly configured to facilitate alignment of fixation devices relative to a bone plate.

FIG. 21 is a perspective view of an exemplary pin inserter.

FIG. 22 is a cross-sectional view of a portion of an example carrier relative to a portion of an example bone plate, and in particular illustrates an engagement tab of the carrier.

FIG. 23 is a top view of an exemplary bone plate.

FIG. 24 is a top view of an exemplary carrier.

FIG. 25 is a cross-sectional view illustrating an example arrangement of a fixation device relative to the carrier.

FIG. 26 illustrates another exemplary fixation device in a relaxed position.

FIG. 27 illustrates the fixation device of FIG. 26 in a deployed position.

FIG. 28 illustrates another exemplary fixation device in a relaxed position.

FIG. 29 illustrates the fixation device of FIG. 28 in a deployed position.

FIG. 30 illustrates an exemplary locking ferrule.

FIG. 31 is a top view of the locking ferrule of FIG. 30.

FIG. 32 illustrates an exemplary fixation device deployment assembly configured for deploying fixation devices relative to a bone plate.

FIG. 33 is an enlarged cross-sectional view of select portions of the fixation device deployment assembly of FIG. 32.

FIGS. 34 and 35 illustrate an exemplary bone plate holding device.

FIG. 36 illustrates another exemplary bone plate holding device.

FIG. 37 illustrates another exemplary bone plate holding device.

FIG. 38 illustrates yet another exemplary bone plate holding device.

### DETAILED DESCRIPTION

This disclosure relates to sternal closure systems for closing a separated sternum. An exemplary sternal closure system may include one or more cerclages formed by suture, one or more bone plates, and a plurality of fixation devices for fixating the bone plate to bone.

An exemplary sternal closure system may include, inter alia, a bone plate including an opening, and a fixation device deployment assembly including a locking tab, a molly anchor, and a deployment pin. When the locking tab is engaged to the fixation device deployment assembly, the fixation device deployment assembly is configured to insert the molly anchor through the opening of the bone plate. When the locking tab is disengaged from the fixation device deployment assembly, the fixation device deployment assembly is configured to cause the deployment pin to move the molly anchor from a relaxed position to a deployed position.

In any further embodiment, the fixation device deployment assembly includes a pin inserter and a pusher having a shaft that is receivable within a through-bore of the pin inserter.

In any further embodiment, the through-bore extends along a longitudinal axis of the pin inserter and extends through both a handle portion and a shaft portion of the pin inserter.

In any further embodiment, the shaft portion of the pin inserter is accommodated within a cannulation of the molly anchor at a location between a first head portion of the molly anchor and a second head portion of the deployment pin.

In any further embodiment, the deployment pin is preassembled to the molly anchor such that the second head portion of the deployment pin is countersunk within the cannulation relative to the first head portion.

In any further embodiment, the second head portion of the deployment pin abuts against a circumferential ledge located within the cannulation of the molly anchor when the molly anchor is in the relaxed position.

In any further embodiment, when the locking tab is disengaged from the shaft of the pusher, the deployment pin is movable into engagement with the molly anchor to cause a first deployable leg and a second deployable leg of the molly anchor to splay apart and thereby transition the molly anchor from the relaxed position to the deployed position.

In any further embodiment, the shaft of the pusher extends from a proximal end section of the pusher, and further wherein the proximal end section includes first diameter that is greater than a second diameter of the shaft.

In any further embodiment, the locking tab is configured to engage the shaft of the pusher to prevent the shaft from engaging the deployment pin.

In any further embodiment, when the locking tab is disengaged from the shaft of the pusher, the deployment pin is movable into engagement with the molly anchor to cause a first deployable leg and a second deployable leg of the molly anchor to splay apart and thereby transition the molly anchor from the relaxed position to the deployed position.

In any further embodiment, the deployment pin is movably accommodated within a cannulation of the molly anchor.

In any further embodiment, the molly anchor includes a head portion and a body portion having a first deployable leg and a second deployable leg that each extend distally from the head portion.

In any further embodiment, the bone plate includes a body including a first longitudinal section, a second longitudinal section, a bridging section extending between the first longitudinal section and the second longitudinal section, a bone contacting surface, and an outer surface opposite of the bone contacting surface. A ridge protrudes from the bone contacting surface and is configured to align and center the bone plate relative to a bone.

In any further embodiment, the bone plate includes a body having a first longitudinal section, a second longitudinal section, a bridging section extending between the first longitudinal section and the second longitudinal section, a bone contacting surface, an outer surface opposite of the bone contacting surface, a first longitudinal slot provided within the first longitudinal section, and a second longitudinal slot provided within the second longitudinal section.

In any further embodiment, a holding device including a holding fork or a holding ball is configured to engage the bone plate for holding the bone plate down against a bone.

An exemplary surgical method may include, inter alia, positioning a bone plate relative to a sternum, advancing a molly anchor of a fixation device deployment assembly through an opening of the bone plate and into the sternum while a locking tab is secured to the fixation device deployment assembly, removing the locking tab from the fixation device deployment assembly, and advancing a deployment pin to cause the molly anchor to transition from a relaxed position to a deployed position relative to the sternum.

In any further embodiment, advancing the molly anchor includes applying a first force to a pusher of the fixation device deployment assembly with the locking tab secured to a shaft of the pusher.

In any further embodiment, advancing the deployment pin includes applying a second force to the pusher with the locking tab removed from the shaft of the pusher.

In any further embodiment, applying the second force advances the deployment pin distally into a cannulation of the molly anchor to release the molly anchor from the fixation device deployment assembly.

In any further embodiment, advancement of the deployment pin distally into the cannulation causes a first deployable leg and a second deployable leg of the molly anchor to splay apart and thereby transition the molly anchor from the relaxed position to the deployed position.

An exemplary method may include, inter alia, advancing a molly anchor of a fixation device deployment assembly through an opening of a bone plate while a locking tab is secured to the fixation device deployment assembly, removing the locking tab from the fixation device deployment assembly, and advancing a deployment pin to cause the molly anchor to transition from a relaxed position to a deployed position relative to the bone plate.

Another exemplary sternal closure system may include, inter alia, a bone plate including a body having a first longitudinal section, a second longitudinal section, a bridging section extending between the first longitudinal section and the second longitudinal section, a bone contacting surface, and an outer surface opposite of the bone contacting surface. A ridge protrudes from the bone contacting surface and is configured to align and center the bone plate between a first sternum bone section and a second sternum bone section of a sternum.

Another exemplary sternal closure system may include, inter alia, a bone plate including a body having a first longitudinal section, a second longitudinal section, a bridging section extending between the first longitudinal section and the second longitudinal section, a bone contacting surface, and an outer surface opposite of the bone contacting surface. A first longitudinal slot is provided within the first longitudinal section, and a second longitudinal slot is provided within the second longitudinal section.

Another exemplary sternal closure system may include, inter alia, a bone plate including a through-passage, a locking ferrule received within the through-passage and including a one-way locking mechanism, and a cerclage including a suture received through the locking ferrule and locked relative to the bone plate by the one-way locking mechanism.

Another exemplary sternal closure system may include, inter alia, a bone plate including an opening, a screw received within the opening and including a head portion, a threaded portion, and a cross-pin mounted within the head portion, and a suture looped around the cross-pin and configured to secure a tissue relative to the bone plate.

Another example sternal closure system may include, inter alia, a bone plate including an opening, and a carrier configured to selectively attach to and detach from the bone plate. The carrier may be configured to temporarily hold a fixation device relative to the opening such that, when the carrier is attached to the bone plate, the fixation device vertically overlaps the opening. Further, upon application of force, the fixation device is configured to release from the carrier and at least partially pass through the opening to fix the bone plate relative to a sternum.

Another example sternal closure system may include, inter alia, a bone plate including an opening, and a fixation device configured to fix the bone plate relative to a sternum. The fixation device may be configurable between a relaxed position and a deployed position. Further, the fixation device may include first and second deployable legs on opposite sides of a slit. The first deployable leg may be longer than the second deployable leg.

Another example sternal closure system may include, inter alia, a bone plate including an opening, and a fixation device configured to fix the bone plate relative to a sternum. The fixation device may be configurable between a relaxed position and a deployed position. Further, the fixation device may include a molly anchor and a wedge anchor, and the wedge anchor may include an eyelet coupled to suture.

Another example sternal closure system may include, inter alia, a bone plate including an opening, and a pin inserter including at least one pin. Further, the at least one pin may be configured to be selectively advanced out of the pin inserter. The system may further include a fixation device configured to fix the bone plate relative to a sternum. Further, the fixation device may be configurable between a relaxed position and a deployed position. The fixation device may include a molly anchor, and the molly anchor may be moveable from the relaxed position to the deployed position as the at least one pin comes into contact with first and second deployable legs of the molly anchor.

Yet another example sternal closure system may include, inter alia, a bone plate including an opening, and an assembly including a pin inserter, a pusher, a locking tab, and a fixation device that includes a molly anchor and a deployment pin. When the locking tab is engaged with a shaft of the pusher, the assembly is configured to insert the molly anchor through the opening, and when the locking tab is disengaged from the shaft, the assembly is configured to allow the deployment pin to move the molly anchor from a relaxed position to a deployed position.

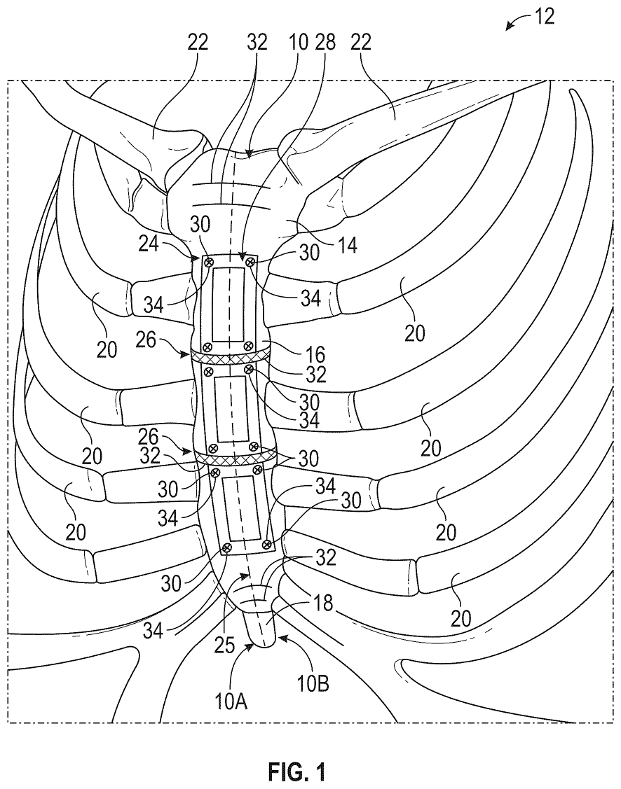

FIG. 1 schematically illustrates a sternum 10 of a human thorax or chest 12. The sternum 10 provides the anterior central part of the chest 12 and includes a manubrium 14, a body 16, and a xiphoid process 18. A plurality of ribs 20 of the chest 12 are either directly or indirectly attached to the sternum 10 and can articulate relative thereto. Clavicles 22 are supported by and can articulate relative to the manubrium 14 of the sternum 10.

As schematically indicated by incision line 25, a sternotomy has previously been performed on the sternum 10 to access organs and/or other tissues located within a patient's chest cavity, such as during a heart surgery, for example. Although described herein in the context of heart surgery, other surgical procedures could necessitate access to the chest cavity, including but not limited to lung surgeries, for example. As a result of the sternotomy, the sternum 10 is divided along the incision line 25 into two sternum bone sections 10A and 10B. The separated sternum bone sections 10A, 10B must be reapproximated relative to one another and then rejoined together after completing the heart surgery.

The separated sternum bone sections 10A and 10B may be reapproximated and then rejoined by a sternal closure system 24. The sternal closure system 24 may include one or more cerclages 26, one or more bone plates 28, and a plurality of fixation devices 30.

Each cerclage 26 may be established by one or more stands of suture 32 that can be arranged to encircle the sternum 10. In an embodiment, the sutures 32 are strands of suture tape, such as FiberTape®, for example. FiberTape® is a suture product marketed and sold by Arthrex, Inc. However, other types of suture could be utilized for the sutures 32 within the scope of this disclosure.

The one or more sutures 32 may be passed through and/or be wrapped around the sternum bone sections 10A and 10B to form the cerclage 26. The sutures 32 may be wrapped around the sternum 10 in various patterns, including figure-8 patterns, for example, to form the cerclage 26. Moreover, as further detailed below, the sutures 32 can be arranged to interface with the bone plate 28 of the sternal closure system 24.

The bone plate 28 may be positioned to straddle the incision line 25 on the sternum 10 such that a first portion of the bone plate 28 interfaces with the sternum bone section 10A and a second portion of the bone plate 28 interfaces with the sternum bone section 10B. The bone plate 28 may include a plurality of openings 34 each configured to receive one of the fixation devices 30. Each fixation device 30 may be inserted through one of the openings 34 and into either the sternum bone section 10A or the sternum bone section 10B for securing the bone plate 28 to the sternum 10. The fixation devices 30 may include screws, anchors, or a combination of screws and anchors. Together, the cerclage 26 and the fixated bone plate 28 cooperate to rejoin the sternum bone sections 10A and 10B together.

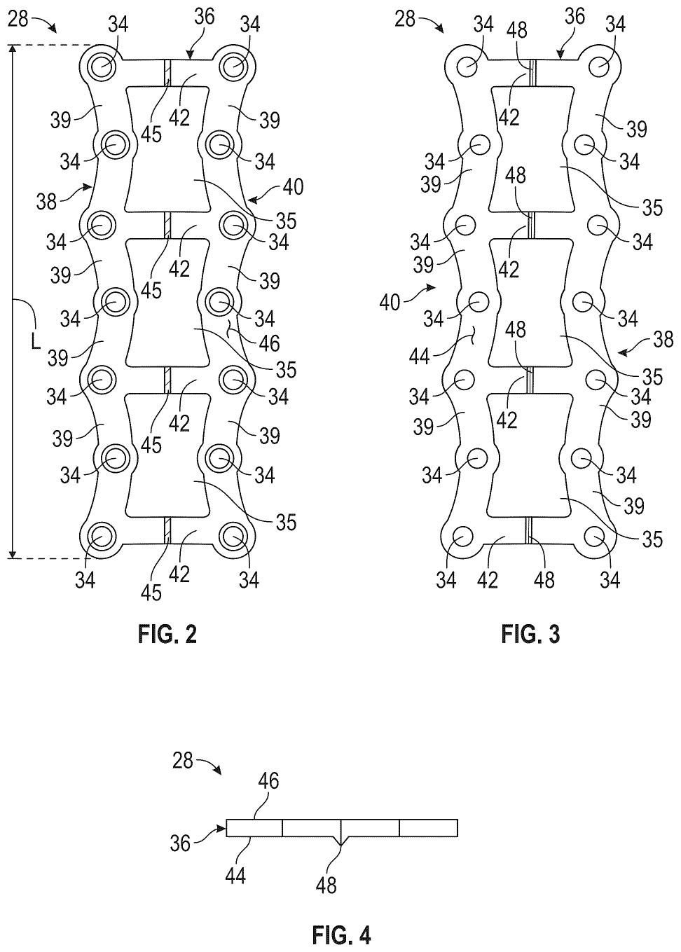

FIGS. 2-4 illustrate an exemplary bone plate 28 that can be utilized as part of the sternal closure system 24 of FIG. 1. The bone plate 28 may be specifically sized and shaped for use relative to the sternum 10. The bone plate 28 may be provided in various sizes for accommodating varying patient sizes.

The bone plate 28 may include a length L. In an embodiment, the length L may be sized to extend across a majority of the superior-to-inferior length of the sternum 10. In another embodiment, the length L may be sized for fixation on a subsection of the sternum 10, such as the manubrium 14 or the body 16, for example.

The bone plate 28 includes a body 36 that can be made from a polyether ether ketone (“PEEK”) material. The PEEK material may enable the body 36 of the bone plate 28 to be anatomically contoured to a particular patient's anatomy. However, the body 36 could be formed from other biocompatible materials or combination of biocompatible materials.

The body 36 includes a first longitudinal section 38, a second longitudinal section 40, and one or more bridging sections 42 that transversely connect between the first and second longitudinal sections 38, 40. When the body plate 28 is received relative to the sternum 10 in the manner shown in FIG. 1, the first longitudinal section 38 interfaces with the sternum bone section 10A, and the second longitudinal section 40 interfaces with the sternum bone section 10B. A cut-out 35 may extend between adjacent bridging sections 42 of the body 36.

Each of the first longitudinal section 38 and the second longitudinal section 40 may include a plurality of subsections 39. In an embodiment, the subsections 39 are curved sections of the first and second longitudinal sections 38, 40. However, in other implementations, the subsections 39 could be substantially straight across their respective lengths. The bridging sections 42 could also be either straight or curved along their respective lengths.

The body 36 of the bone plate 28 may further include a bone contacting surface 44 and an outer surface 46 on an opposite side of the bone plate 28 from the bone contacting surface 44. The bone contacting surface 44 interfaces with the sternum 10 when the bone plate 28 is positioned thereon.

The bone contacting surface 44 may include one or more ridges 48 (see FIGS. 3 and 4) that are configured to be placed along the incision line 25 for aligning and centering the bone plate 28 between the sternum bone sections 10A, 10B. One or more of the ridges 48 may extend outwardly from each bridging section 42 of the body 36 and can protrude in a direction away from the outer surface 46.

The outer surface 46 may include one or more laser lines 45 that are axially aligned with the ridges 48. The laser lines 45 provide visual cues for aligning and centering the bone plate 28 to the incision line 25.

A first portion of the openings 34 of the bone plate 28 may be provided within the first longitudinal section 38 of the body 36, and a second portion of the openings 34 of the bone plate 28 may be provided within the second longitudinal section 40 of the body 36. The openings 34 extend completely through the body 36 and therefore open through both the bone contacting surface 44 and the outer surface 46. In an embodiment, the openings 34 are equally sized openings that are either aligned or staggered relative to one another. However, other configurations are also contemplated, and thus the total number of openings 34 and their specific arrangement within the body 36 are not intended to limit this disclosure.

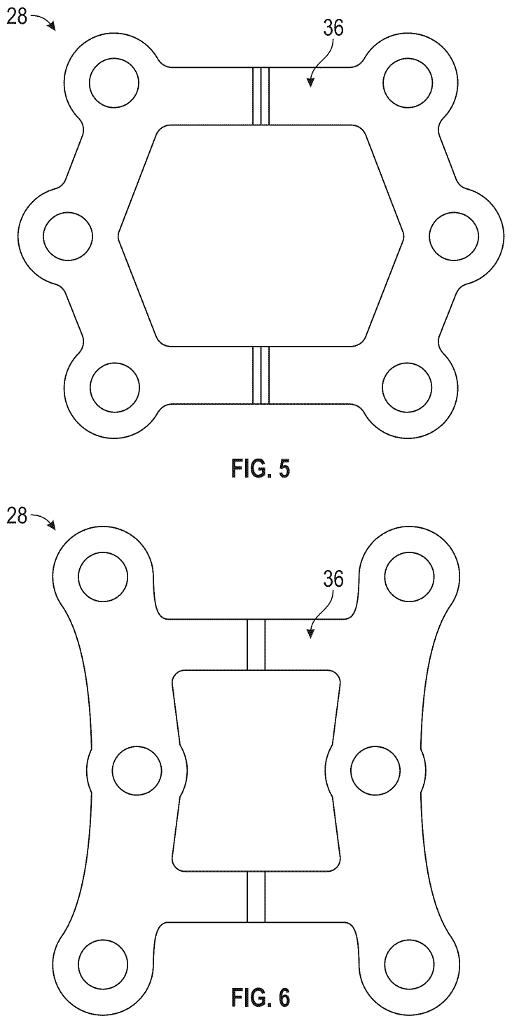

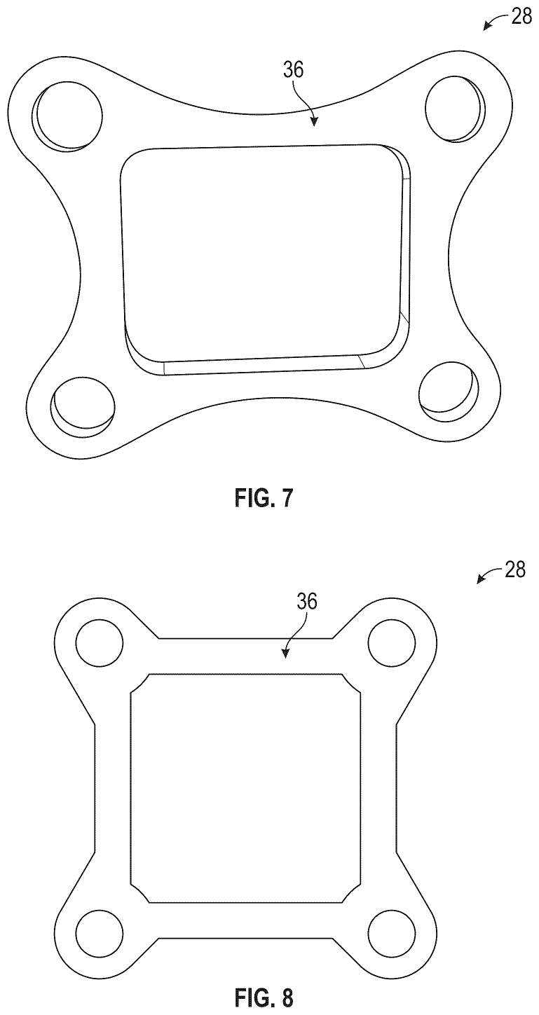

The body 36 of the bone plate 28 shown in FIGS. 2-4 includes an infinity or figure-8-like shape. However, other shapes are contemplated within the scope of this disclosure. For example, the body 36 of the bone plate 28 could include a hexagonal shape (see FIG. 5), an H-shape (see FIG. 6), a rectangular shape (see FIG. 7), a square shape (see FIG. 8), or any other shape that is suitable to be mounted to the sternum 10 or any subsection thereof.

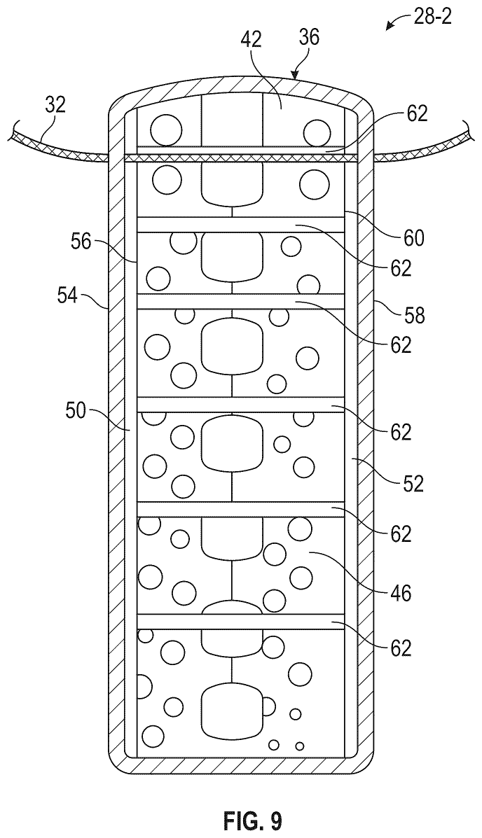

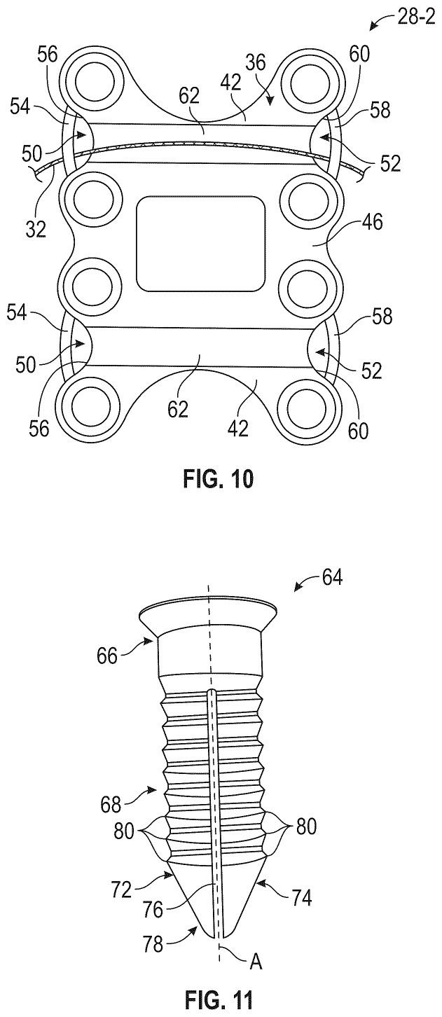

FIGS. 9-10 illustrate another exemplary bone plate 28-2. The bone plate 28-2 may include all the features described above with respect to the bone plate 28 of FIGS. 2-4. The bone plate 28-2 may additionally include a first elongated slot 50 and a second elongated slot 52 formed through the body 36. The first elongated slot 50 may be disposed between a first outboard rail 54 of the body 36 and a first lateral edge 56 of the first longitudinal section 38, and the second elongated slot 52 may be disposed between a second outboard rail 58 of the body 36 and a second lateral edge 60 of the second longitudinal section 40. In an embodiment, the first elongated slot 50 and the second elongated slot 52 extend across a majority of the length of the body 36 (see FIG. 9). In another embodiment, the body 36 may include two or more first elongated slots 50 and two or more second elongated slots 52 (see FIG. 10).

One or more channels 62 may be formed in the outer surface 46 of the body 36. The channels 62 may extend from the first elongated slot 50 to the second elongated slot 52 and may each bisect transversely across one of the bridging sections 42. Each channel 62 may thus establish a countersunk region of the outer surface 46.

The first elongated slot 50, the second elongated slot 52, and the channels 62 may cooperate to allow the one or more sutures 32 of the cerclage 26 to interface with the bone plate 28. For example, each suture 32 may be shuttled through the first elongated slot 50, across one of the channels 62, and through the second elongated slot 52 while forming the cerclage 26 about the sternum 10. The sutures 32 may slide within the first and second elongated slots 50, 52 to allow the bone plate 28 to be adjusted in a superior direction or an inferior direction relative to the sternum 10 prior to final fixation of the bone plate 28. Further, the sutures 32 may provide sufficient tension to hold the bone plate 28 in place before securely fixating the bone plate 28 to the sternum 10 with the fixation devices 30. The sutures 32 may be tensioned simultaneously with inserting the fixation devices 30 to achieve definitive fixation of the bone plate 28 to the sternum 10.

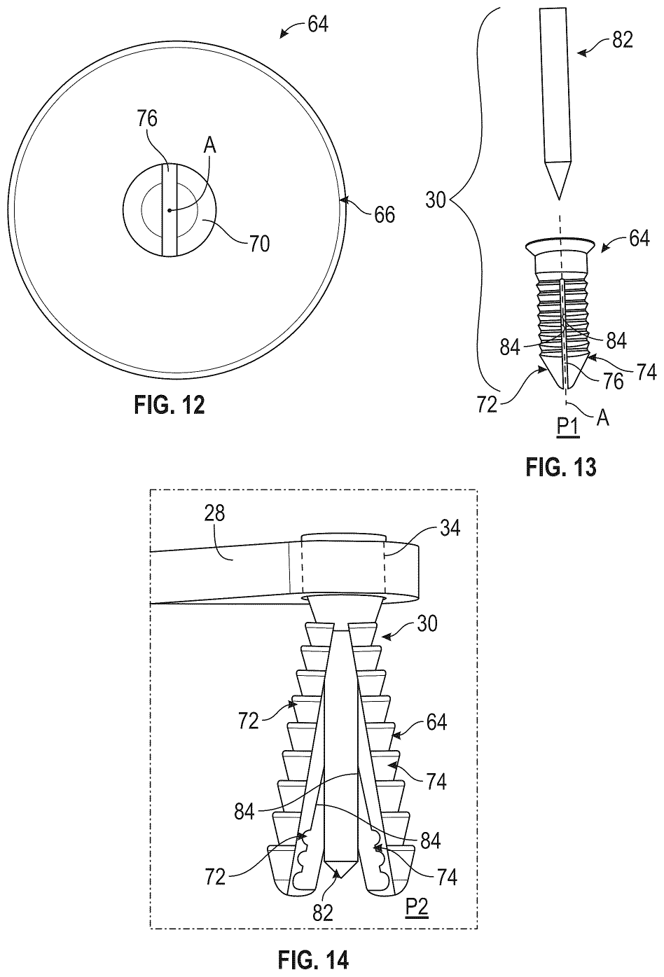

FIGS. 11-14 illustrate features associated with an exemplary fixation device 30 that can be utilized as part of the sternal closure system 24 of FIG. 1 for securing the bone plate 28 down on the sternum 10. In an embodiment, the fixation device 30 is made from the same type of material as the bone plate 28 (e.g., a PEEK material). However, the fixation device 30 could alternatively be constructed out of a different material than the bone plate 28.

The fixation device 30 may include a molly anchor 64 that extends along a longitudinal axis A between a head portion 66, located proximally, and a body portion 68, located distally. The head portion 66 may be configured to mate with one of the openings 34 of the bone plate 28 when the molly anchor 64 is inserted therethrough. A cannulation 70 may extend through the head portion 66 and may circumscribe the longitudinal axis A.

The body portion 68 of the molly anchor 64 may include a first deployable leg 72 and a second deployable leg 74. The first deployable leg 72 and the second deployable leg 74 may be separated from one another by a slit 76 that is formed in the body portion 68. The slit 76 may extend from the head portion 66 to a distal tip 78 of the body portion 68. The cannulation 70 of the head portion 66 may open into the slit 76.

The first deployable leg 72 and the second deployable leg 74 may each include a plurality of bone engagement features 80 for facilitating bone fixation once the molly anchor 64 is inserted through the bone plate 28 and into the sternum 10. In an embodiment, the bone engagement features 80 include barbs or ledges. In another embodiment, the bone engagement features 80 include screw-like threads. However, other configurations are also contemplated within the scope of this disclosure.

Referring now primarily to FIGS. 13-14, the fixation device 30 may further include a deployment pin 82 that is configured for moving the first and second deployable legs 72, 74 of the molly anchor 64 between a relaxed position P1 (see FIG. 13) and a deployed position P2 (see FIG. 14). The molly anchor 64 includes a greater footprint when in the deployed position P2 compared to the relaxed position P1. In an embodiment, the deployment pin 82 is made of a PEEK material.

During implantation, the deployment pin 82 may be inserted through the cannulation 70 of the head portion 66. Upon exiting the cannulation 70, the deployment pin 82 may enter the slit 76 and then engage an inner wall 84 of each of the first and second deployable legs 72, 74. As the deployment pin 82 is moved further distally within the slit 76, the deployment pin 82 forces the first and second deployable legs 72, 74 to splay apart from one another in a radially outward direction, thereby moving the legs from the relaxed position P1 to the deployed position P2. In the deployed position P2, the first and second deployable legs 72, 74 are positioned to engage the bone and prevent the fixation device 30 from backing out of the opening 34 of the bone plate 28.

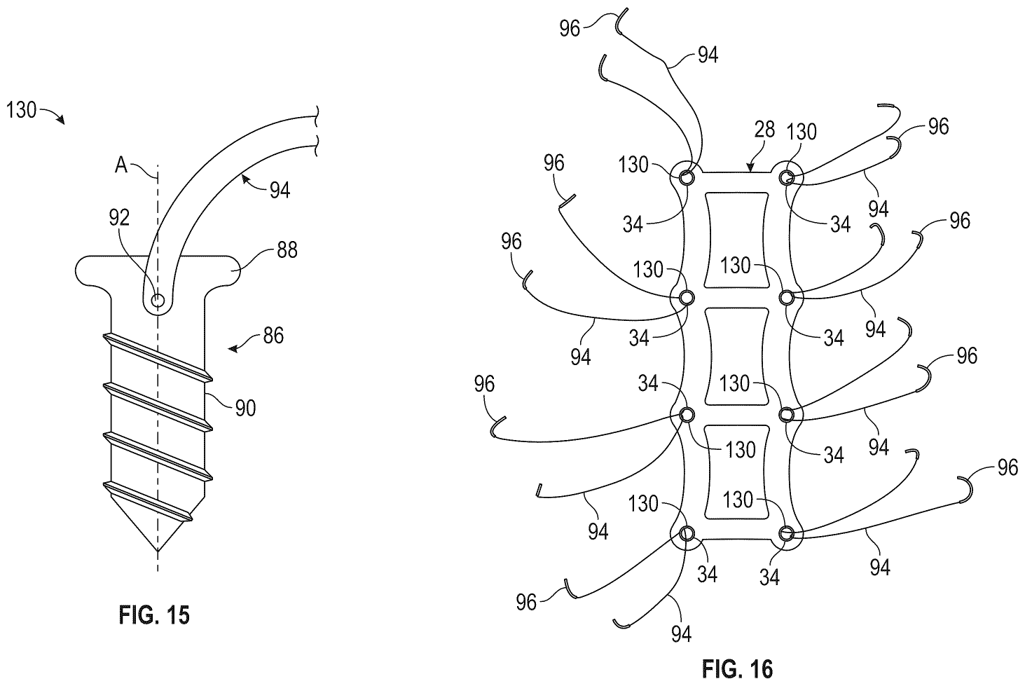

FIGS. 15-16 illustrate another exemplary fixation device 130 that can be utilized as part of the sternal closure system 24 of FIG. 1 for securing the bone plate 28 down on the sternum 10. The fixation device 130 may include a screw 86 that extends along a longitudinal axis A between a head portion 88 and a threaded body 90. The head portion 88 may be configured to mate with one of the openings 34 of the bone plate 28, and the threaded body 90 may be screwed into underlying bone when the screw 86 is inserted therethrough.

A cross-pin 92 may be mounted within the head portion 88 of the screw 86. A suture 94 of the fixation device 130 may be looped around the cross-pin 92. A needle 96 may be attached to free ends of the suture 94. The needle 96 may be utilized to pierce through various tissues (e.g., sternopericardial ligament, sternocleidomastoid muscle, etc.) associated with the sternum 10, and the suture 94 may then be used for securing the tissue relative to the bone plate 28 during the sternal closure.

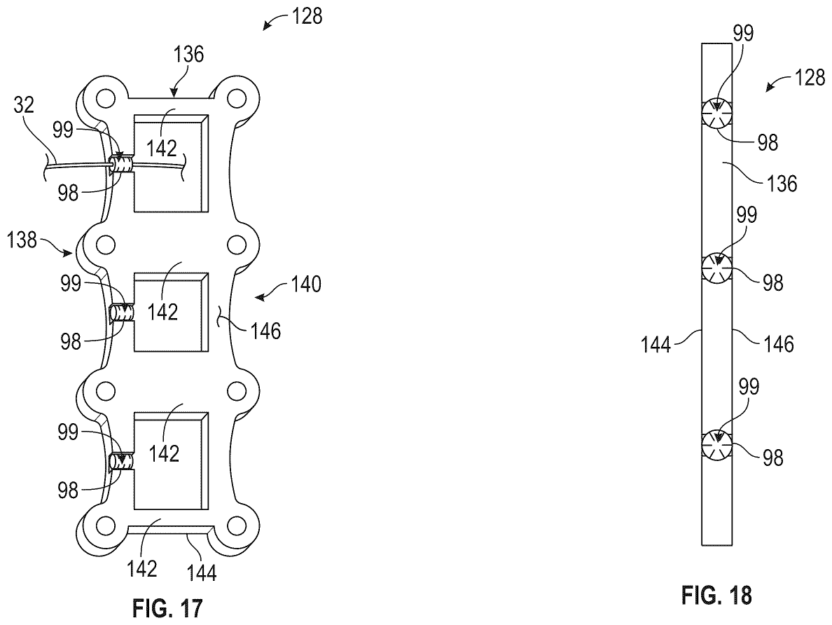

FIGS. 17-18 illustrate another exemplary bone plate 128 that can be utilized as part of the sternal closure system 24 of FIG. 1. The bone plate 128 includes a body 136 that includes a first longitudinal section 138, a second longitudinal section 140, and one or more bridging sections 142 that transversely connect between the first and second longitudinal sections 138, 140. The body 136 further includes a bone contacting surface 144 and an outer surface 146 on an opposite side of the bone plate 128 from the bone contacting surface 144.

Through-passages 98 may be formed through one or both of the first longitudinal section 138 and the second longitudinal section 140. Each through-passage 98 may be sized to receive a locking ferrule 99. The locking ferrules 99 may each be configured for tensioning and knotlessly locking one or more sutures 32 when forming the cerclage 26 around the sternum 10. Other implementations could involve positioning the through-passages 98 and the locking ferrules 99 within the bridging sections 142 of the body 136, for example.

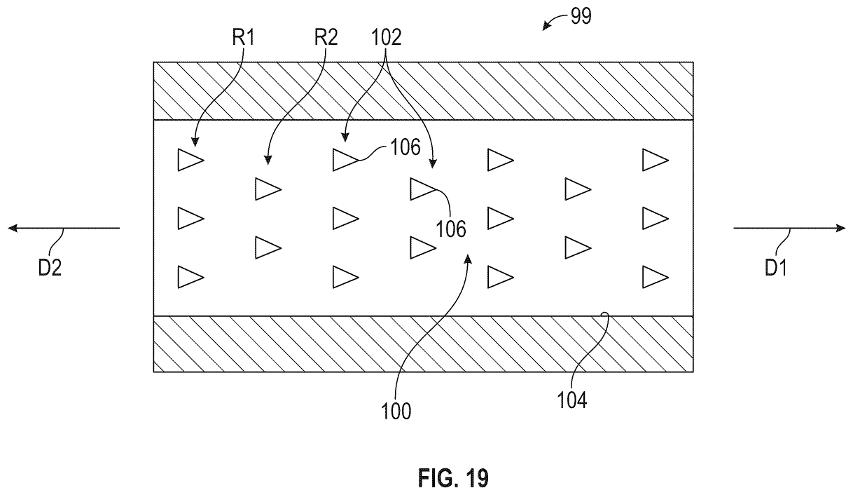

As best illustrated in FIG. 19, each locking ferrule 99 may include a cannulation 100 that establishes an internal passageway for accommodating one or more strands of the suture 32. A plurality of locking barbs 102 may protrude inwardly from an inner diameter wall 104 that circumscribes the cannulation 100. The locking barbs 102 may therefore occupy at least a portion of the open space of the cannulation 100.

The locking barbs 102 may be arranged in multiple rows along the length of the cannulation 100. For example, the locking barbs 102 may be arranged in a least a first row R1 and a second row R2. In an embodiment, the locking barbs 102 of the second row R2 are staggered relative to the locking barbs 102 of the first row R1.

Each locking barb 102 may include a sharp or pointed tip 106, and each locking barb 102 may be angled in a common direction. The locking barbs 102 may therefore establish a one-way locking mechanism that permits the suture(s) 32 to pass through the cannulation 100 in a first direction D1 while preventing the suture(s) 32 from being tensioned or otherwise moved in a second direction D2. The staggered relationship of the rows of locking barbs 102 may provide for maximum engagement with the suture 32 one it is passed through the cannulation 100.

During the sternal closure, the suture(s) 32 may be wrapped around the sternum 10 and then passed through the cannulation 100 of the locking ferrule 99. The suture(s) 32 may then be tensioned to lock the suture(s) 32 in place and thereby form the cerclage 26 around the sternum 10.

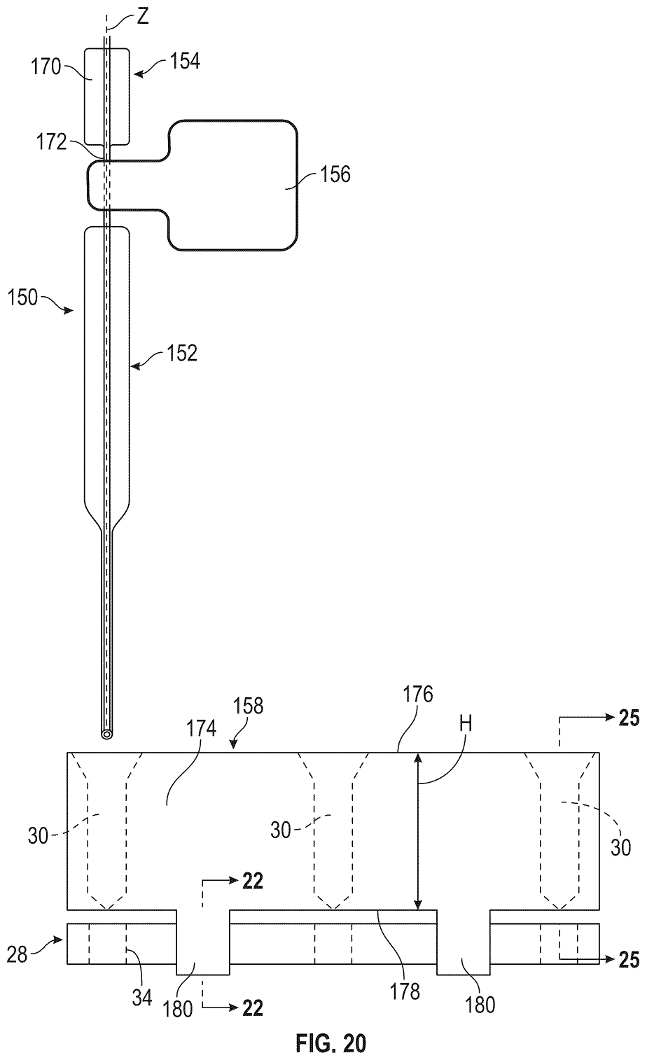

FIG. 20 illustrates an assembly 150 configured to facilitate deployment of fixation devices 30 relative to the bone plate 28. Parts of the assembly 150 can be used as part of the sternal closure system 24 of FIG. 1 for securing the bone plate 28 relative to the sternum 10. While bone plate 28 and fixation devices 30 are shown in FIG. 20, the assembly 150 can be used with the bone plate 128, fixation devices 130, and other bone plates or fixation devices.

In this example, the assembly 150 includes a pin inserter 152, a pusher 154, a locking tab 156, and a carrier 158. The fixation devices 30 and the bone plate 28 may be considered part of the assembly 150.

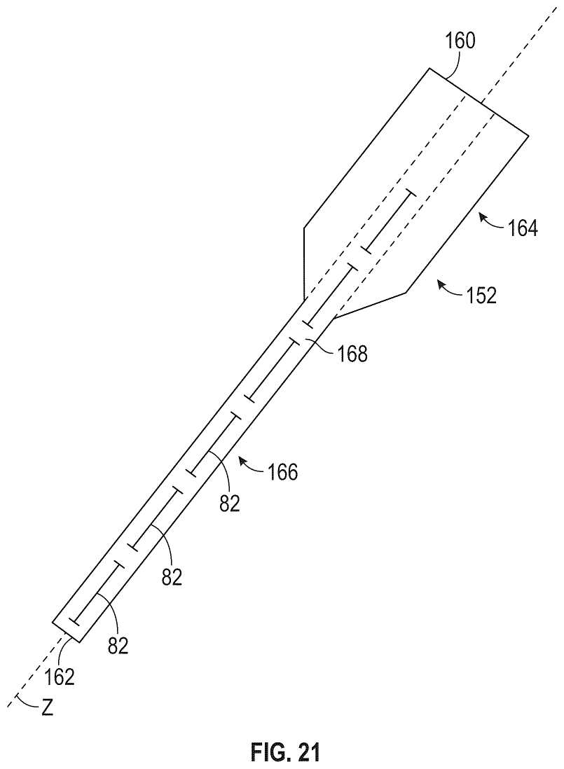

The pin inserter 152 is shown in greater detail in FIG. 21, which illustrates the pin inserter 152 in cross-section. The pin inserter 152 extends along an axis Z between a proximal end 160 and a distal end 162. The pin inserter 152 may include a handle portion 164 adjacent the proximal end 160, and may further include a shaft portion 166 projecting from the handle portion 164 and extending from the handle portion 164 to the distal end 162. The shaft portion 166 exhibits a lesser diameter than the handle portion 164. The handle portion 164 may be sized and shaped so as to be comfortably gripped by a user. The pin inserter 152 may be fully cannulated, including a through-bore 168 extending from the proximal end 160 to the distal end 162. In this example, the through-bore 168 is loaded with a plurality of deployment pins 82. While a particular number of deployment pins 82 are shown in FIG. 21, this disclosure is not limited to a particular number of deployment pins 82.

With reference back to FIG. 20, the pusher 154 includes a proximal end section 170 and a shaft 172 projecting distally from the proximal end section 170. The proximal end section 170 exhibits a greater diameter than the shaft 172. The shaft 172 may be sized and shaped to enter into the through-bore 168 and to engage a proximal-most one of the deployment pins 82 to advance them distally within the through-bore 168 and to eject the distal-most one of the deployment pins 82 from the through-bore 168. The proximal end section 170 is sized and shaped so as to facilitate and endure a transfer of force from another object, such as a mallet. The locking tab 156 may be configured to selectively engage and disengage a portion of the shaft 172 projecting distally of the pin inserter 152 to prevent unwanted advancement of the pusher 154.

The carrier 158 may be configured to support the fixation devices 30 relative to the bone plate 28 so as to facilitate alignment of the fixation devices 30 relative to the bone plate 28 and facilitate advancement of the fixation devices 30 into the sternum, in this example.

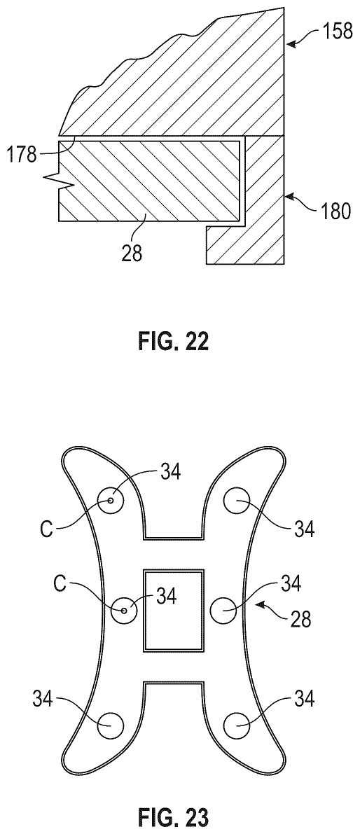

The carrier 158, in this example, includes a main housing section 174 including a top surface 176 and a bottom surface 178 spaced-apart from one another by a height H. When mounted to a bone plate 28 arranged relative to sternum 10, the top surface 176 may be referred to as an anterior surface, and the bottom surface 178 may be referred to as a posterior surface. The height H may be substantially equal to a length dimension of the fixation devices 30. The carrier 158 further includes engagement tabs 180, which in this example project in a downward direction relative to the bottom surface 178. The engagement tabs 180 are configured to snap around an outer edge of the bone plate 28 to selectively attach the carrier 158 to the bone plate 28, as generally shown in FIG. 22. The engagement tabs 180 may be arranged about the perimeter of the carrier 158 so as to facilitate alignment of the carrier 158 relative to the bone plate 28. While engagement tabs 180 are shown and described, this disclosure extends to other techniques for aligning the carrier 158 relative to the bone plate 28.

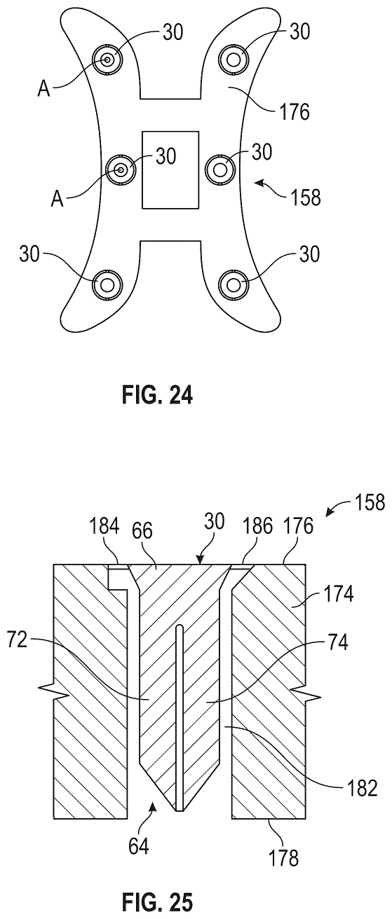

As seen in FIGS. 23 and 24, the carrier 158 exhibits a shape substantially similar to that of the bone plate 28. While a particular configuration of the bone plate 28, and a corresponding configuration of the carrier 158, is shown in FIGS. 23 and 24, this disclosure extends to other bone plate configurations, and other corresponding carriers, which would be sized and shaped to correspond to the particular bone plate.

The bone plate 28 includes six openings 34, in this example. When the carrier 158 is aligned such that the carrier 158 vertically overlaps the bone plate 28, namely when the tabs 180 are engaged with the bone plate 28, the fixation devices 30 are arranged above a corresponding one of the openings 34. Namely, axes A of the fixation devices 30 are substantially concentric with a centerline C of a corresponding opening 34. As shown in FIG. 24, there are six fixation devices 30, in this example, with each one corresponding to one of the openings 34.

With reference to FIG. 25, an example configuration of the carrier 158 relative to one of the fixation devices 30 is shown. The carrier 158 may be configured similarly relative to the other fixation devices 30. In this example, the carrier 158 may be configured to selectively hold the fixation device 30 such that, upon application of force, the fixation devices 30 will break free of the carrier 158. In this example, the carrier 158 includes a through-bore 182 extending from the top surface 176 to the bottom surface 178. The through-bore 182 may be sized to as to permit the fixation device 30 to pass through the through-bore 182. That said, the through-bore 182 is, in this example, in relatively close proximity to the fixation device 30 to prevent premature movement of the first and second deployable legs 72, 74 to the deployed position. In this example, the fixation device 30 includes the molly anchor 64 of FIGS. 13 and 14. The carrier 158, in this example, includes first and second frangible tabs 184, 186 projecting from the main housing section 174 to the head portion 66 of the molly anchor 64. The first and second frangible tabs 184, 186 are configured to release the head portion 66 of the molly anchor 64 upon application of a sufficient force. While frangible tabs are shown and described, this disclosure encompasses other techniques for temporarily holding the fixation devices 30 by the carrier 158. Further, while the fixation devices 30 are not shown with threads, this disclosure extends to threaded fixation devices. The fixation devices 30 may alternatively include barbs or ledges, as examples.

In an example method of use, a bone plate 28 may be arranged relative to the sternum 10. The carrier 158, with the fixation devices 30 attached thereto, may be attached to the bone plate 28 using the tabs 180, for example. A user may situate the pin inserter 152 relative to a particular one of the fixation devices 30, remove the locking tab 156, and apply a force to the proximal end section 170 of the pusher 154, such as by using a mallet. Doing so advances one of the deployment pins 82 distally out of the pin inserter 152, releases the fixation device 30 from the carrier 158 by fracturing tabs 184, 186, advances the fixation device 30 partially through a corresponding one of the openings 34 of the bone plate 28, and causes the first and second deployable legs 72, 74 to move into the deployed position, as shown in FIG. 14. The process is repeated for the remaining fixation devices 30. The carrier 158 may then be detached from the bone plate 28.

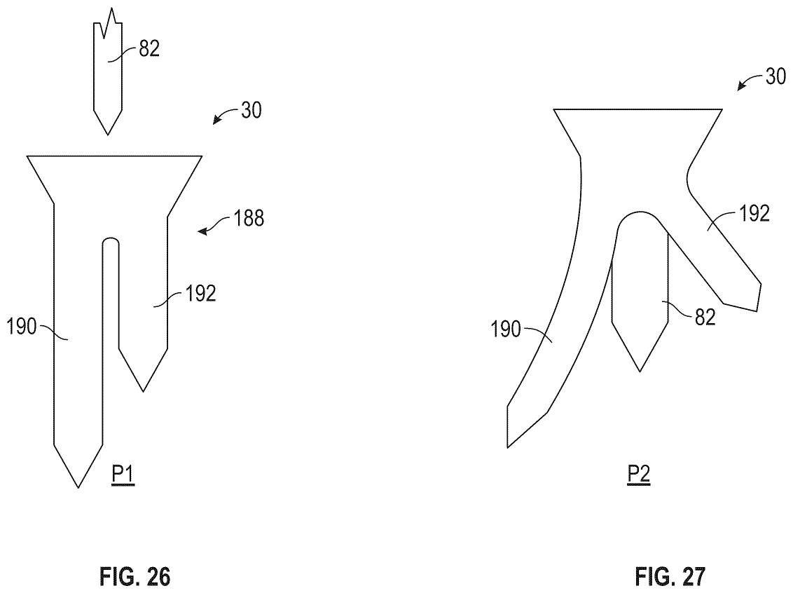

FIGS. 26 and 27 illustrate another example fixation device 30, which may be used with the sternal closure system 24, in the relaxed and deployed positions respectively. In this example, the example fixation device 30 may be a molly anchor 188 and may be configured substantially similar to the molly anchor 64, with the exception that the molly anchor 188 includes first and second deployable legs 190, 192 of different lengths. The first deployable leg 190, which exhibits a greater length than the second deployable leg 192, may facilitate penetration of the molly anchor 188 into bone. In this regard, the molly anchor 188 may be considered self-punching. The second deployable leg 192 may enter into a bone hole formed by the first deployable leg 190. The mismatched length of the first and second deployable legs 190, 192 may provide self-punching functionality while avoiding premature movement of the molly anchor 188 to the deployed position.

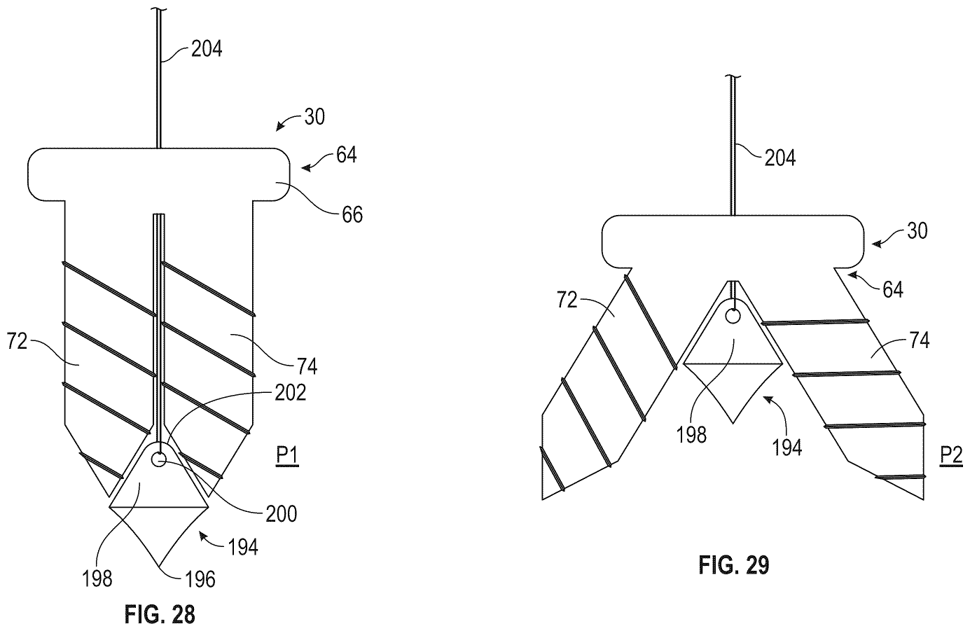

FIGS. 28 and 29 illustrate another example fixation device 30, which may be used with the sternal closure system 24, in the relaxed and deployed positions respectively. In this example, the fixation device 30 includes the molly anchor 64, or an anchor configured substantially similar thereto, and further includes a wedge anchor 194. The wedge anchor 194 may be part of the molly anchor 64 or could be incorporated as part of an inserter device (not shown for simplicity and clarity) usable to implant the molly anchor 64. The molly anchor 64 may be threaded in this example. The fixation device 30 of FIGS. 28 and 29 may be considered self-punching. When the molly anchor 64 is in the relaxed position, the wedge anchor 194 may be arranged adjacent a distal end of the first and second deployable legs 72, 74. The carrier 158 may temporarily hold the wedge anchor 194 in this configuration. Alternatively or additionally, the first and second deployable legs 72, 74 may include one or more features, such as notches or grooves, configured to temporarily hold the wedge anchor 194 in the configuration of FIG. 28.

The wedge anchor 194 includes a substantially sharp distal tip 196 to facilitate penetration into bone. The wedge anchor 194 further includes a wedge section 198 tapering toward an eyelet 200 adjacent a proximal end 202 of the wedge anchor 194. In this example, suture 204 may be threaded through the eyelet 200 and passes through the molly anchor 64 and projects proximally of the head portion 66 of the molly anchor 64 such that the suture 204 can be manipulated by a user. In an example method of use, a force can be applied to the fixation device 30, thereby causing the distal tip 196 to penetrate bone and to cause the first and second deployable legs to advance into a bone hole formed by the distal tip 196. A user can then pull the suture 204, which causes the wedge section 198 to force the first and second deployable legs 72, 74 into the deployed position. With the fixation device 30 in the deployed position of FIG. 29, a user may tie soft tissue and/or skin relative to the bone plate 28 via suture 204. Alternatively or additionally, suture 204 may establish one or more of the cerclages 26. The suture 204 may be made of the same material as suture 32.

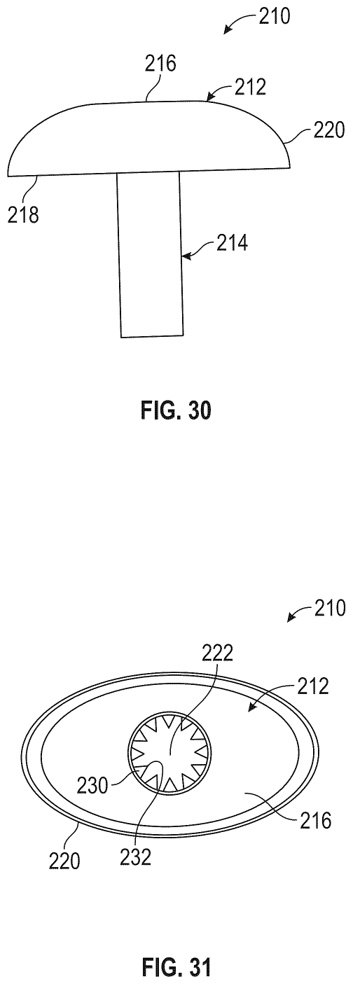

FIGS. 30 and 31 illustrate an exemplary locking ferrule 210 that can be used when performing various tensionable surgical procedures, including using the fixation device of FIGS. 28 and 29, or similar fixation devices. The locking ferrule 210 may be used with the sternal closure system 24. In particular, the locking ferrule 210 may be provided adjacent to or within through passages in a bone plate, similar to the locking ferrule 99 described above.

The locking ferrule 210 may be an exemplary suture locking device that can include a cap portion 212 and a barrel portion 214 that protrudes from the cap portion 212. The cap portion 212 and the barrel portion 214 may be formed together as part of a unitary structure of the locking ferrule 210. The cap portion 212 may rest against outer surface 46 of the bone plate 28, and the barrel portion 214 may project into an opening 34 of the bone plate 28, in an example. In an embodiment, the cap portion 212 and the barrel portion 214 are arranged to provide a top hat-like appearance of the locking ferrule 210. However, the specific size, shape, and material make-up of the locking ferrule 210 are not intended to limit this disclosure.

The cap portion 212 of the locking ferrule 210 may include a top surface 216, a bottom surface 218, and a side wall 220 that extends between the top surface 216 and the bottom surface 218. The barrel portion 214 may protrude outwardly from the bottom surface 218 of the cap portion 212. A cannulation 222 may extend through the cap portion 212 and the barrel portion 214. The cannulation 222 may establish an internal passageway for accommodating one or more strands of suture or other structures (e.g., wires, shafts, etc.). The locking ferrule 210 of this embodiment may therefore provide a “single-chamber” design.

A plurality of locking barbs 230 may occupy at least a portion of the open space of the cannulation 222. In an embodiment, the locking barbs 230 are integrally formed (e.g., molded) features of the body of the locking ferrule 210. The locking barbs 230 may be provided along an entire length of the cannulation 222 or at only select portions thereof. The locking barbs 230 may be either rigid or flexible structures.

Each locking barb 230 may include a sharp or pointed tip 232, and, in this implementation, each locking barb 230 may be angled in a direction toward the cap portion 212. The locking barbs 230 may therefore establish a one-way locking mechanism that permits one or more sutures to pass through the cannulation 222 in a first direction while preventing the suture(s) from being tensioned or otherwise moved in a second, opposite direction.

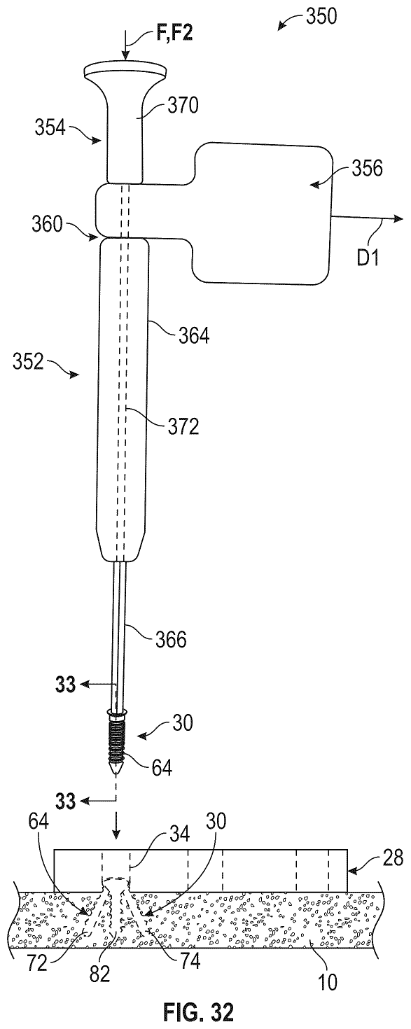

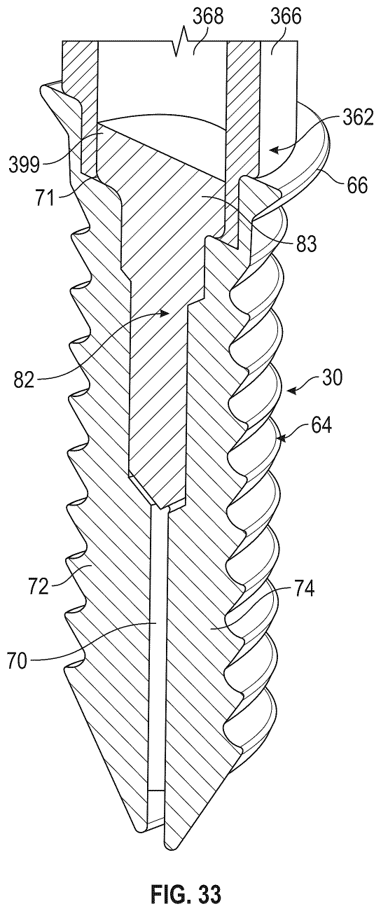

FIGS. 32-33 illustrate another exemplary assembly 350 configured to facilitate deployment of a fixation device 30 relative to the bone plate 28. The assembly 350 may be referred to as a fixation device deployment assembly. In this embodiment, the fixation device 30 may include the molly anchor 64 and the deployment pin 82, and the assembly 350 can be used as part of the sternal closure system 24 of FIG. 1 for securing the bone plate 28 relative to the sternum 10. While the bone plate 28 and the molly anchor 64 are specifically shown in FIG. 32, the assembly 350 can be used with the bone plate 128 and/or other bone plates and/or other fixation devices.

The assembly 350 may include a pin inserter 352, a pusher 354, and a locking tab 356. The fixation device 30, which in this embodiment includes both the molly anchor 64 and the deployment pin 82, may also be considered part of the assembly 350. The bone plate 28 may be utilized with the assembly 350 as part of the sternal closure system 24.

The pin inserter 352 may extend along a longitudinal axis between a proximal end 360 and a distal end 362. The pin inserter 352 may include a handle portion 364 adjacent the proximal end 360 and a shaft portion 366 projecting outwardly from the handle portion 364 to the distal end 362. The shaft portion 366 may exhibit a lesser diameter than the handle portion 364. The handle portion 364 may be sized and shaped so as to be comfortably gripped by a user. The pin inserter 352 may be fully cannulated and may thus include a through-bore 368 extending from the proximal end 360 to the distal end 362.

The fixation device 30 may be pre-loaded onto the distal end 362 of the pin inserter 352. When positioned thereon, a portion 399 (see FIG. 33) of the shaft portion 366 that provides the distal end 362 of the pin inserter 352 may be accommodated within the cannulation 70 of the molly anchor 64 at a location between the head portion 66 of the molly anchor 64 and a head portion 83 of the deployment pin 82. The head portion 66 of the molly anchor 64 may circumscribe an outer diameter surface of the portion 399 of the shaft portion 366 when the fixation device 30 is preassembled to the pin inserter 352. The deployment pin 82 may be preassembled to the molly anchor 64 such that the head portion 83 of the deployment pin 82 is slightly countersunk relative to the head portion 66 of the molly anchor 64. A circumferential ledge 71 (see FIG. 33) located within the cannulation 70 may prevent the shaft portion 366 of the pin inserter 352 and the head portion 83 of the deployment pin 82 from being inserted too far into the cannulation 70 prior to full deployment of the fixation device 30. For example, the head portion 83 of the deployment pin 82 can abut against the circumferential ledge 71 to prevent advancement of the deployment pin 82 further distally into the cannulation 70 prior to full deployment of the fixation device 30.

In the illustrated embodiment, the assembly 350 is configured to deploy a one fixation device 30 at a time. However, other configurations are contemplated within the scope of this disclosure.

The pusher 354 of the assembly 350 may include a proximal end section 370 and a shaft 372 projecting distally from the proximal end section 370. The proximal end section 370 exhibits a greater diameter compared to the shaft 372. The shaft 372 may be sized and shaped for insertion into the through-bore 368 to engage the head portion 83 of the deployment pin 82 for causing the first and second deployable legs 72, 74 of the molly anchor 64 to splay apart from one another after the molly anchor 64 has been inserted into one of the openings 34 of the bone plate 28. The proximal end section 370 may be sized and shaped so as to facilitate and endure a transfer of force from another object, such as a mallet, for example, for forcing the deployment pin 82 further distally into the cannulation 70.

The locking tab 356 may be configured to selectively engage and disengage the shaft 372 of the pusher 354 to prevent unwanted advancement of the pusher 354. When engaged to the shaft 372, the locking tab 356 may be positioned between the proximal end 360 of the pin inserter 352 and the proximal end section 370 of the pusher 354. The shaft 372 cannot engage the head portion 83 of the deployment pin 82 without first disengaging the locking tab 356 from the shaft 372. Once the locking tab 356 is removed from the shaft 372, the shaft 372 can be moved further distally within the through-bore 368 for engaging the head portion 83 of the deployment pin 82.

In an example method of use of the assembly 350, the bone plate 28 may be arranged relative to the sternum 10. The pin inserter 352, with the pusher 354, the locking tab 356, and the fixation device 30 attached thereto, may be positioned relative to one of the openings 34 of the bone plate 28. A force F may then be applied (e.g., with a mallet) to the proximal end section 370 of the pusher 354 with the locking tab 356 secured to the shaft 372 to advance the molly anchor 64 through the opening 34 of the bone plate 28 and into the sternum 10. A pilot hole may be formed (e.g., punched, drilled, etc.) in the sternum 10 prior to inserting the molly anchor 64 through the opening 34.

A user may next remove the locking tab 356 from the shaft 372 (e.g., by pulling in the direction of arrow D1) and then apply an additional force F2 to the proximal end section 370 of the pusher 354, such as by using the mallet. Doing so advances the deployment pin 82 further distally into the cannulation 70, thereby causing the molly anchor 64 to be released from the pin inserter 352 and the first and second deployable legs 72, 74 to move to the deployed position. In the deployed position, the first and second deployable legs 72, 74 can engage the sternum 10 and prevent the molly anchor 64 from backing out of the opening 34 of the bone plate 28. This process may be repeated for inserting and deploying additional fixation devices 30 through the bone plate 28 and into the sternum 10.

Although not specifically shown, a carrier, such as the carrier 158, for example, could be utilized to facilitate alignment of the fixation devices 30 relative to the bone plate 28 prior to insertion of the fixation devices 30 through the bone plate 28. The carrier 158 may also be used to guide the formation of pilot holes that can be drilled or punched into the sternum 10 prior to inserting the molly anchor 64 through the opening 34.

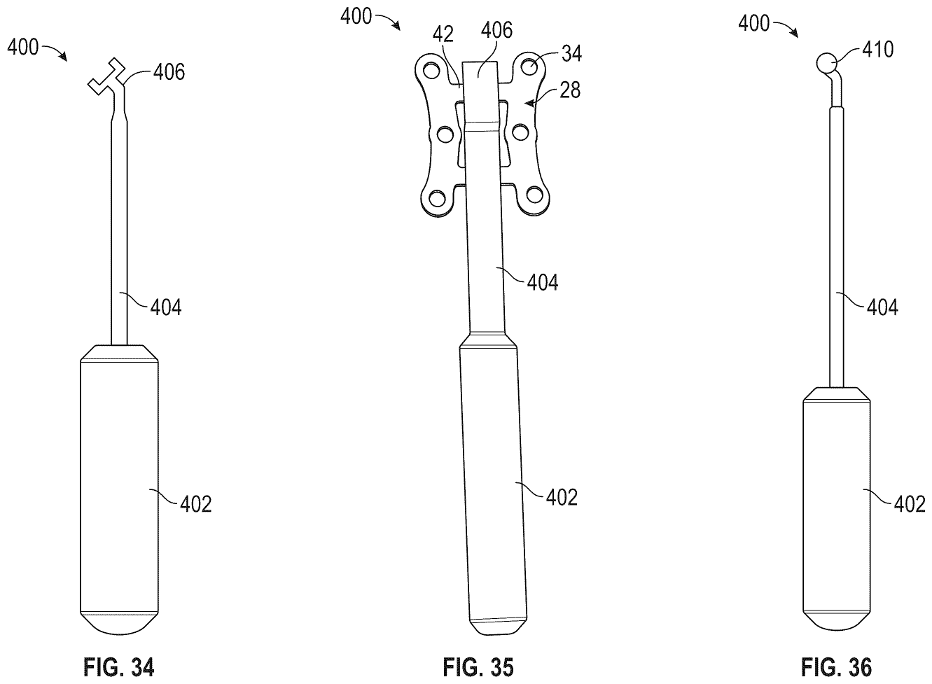

A holding device 400 may be utilized to hold the bone plate 28 in place while inserting the fixation devices 30 in the manner described above. For example, as shown in FIGS. 34 and 35, the holding device 400 may include a handle 402, a shaft 404 projecting from the handle 402, and a holding fork 406 provided at a distal end of the shaft 404. The holding fork 406 may be sized to be arranged about one of the bridging sections 42 of the bone plate 28 for more firmly holding the bone plate 28 down against the sternum 10 when inserting the fixation devices 30.

In another embodiment, shown in FIG. 36, the holding device 400 may include a holding ball 410 instead of the holding fork 406. The holding ball 410 may be sized to be positioned within one of the openings 34 of the bone plate 28 for more firmly holding the bone plate 28 down against the sternum 10 when inserting the fixation devices 30.

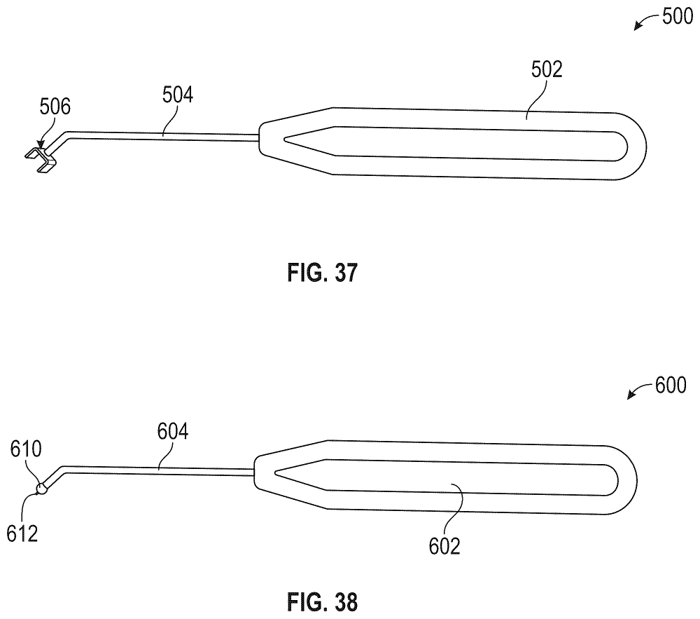

FIG. 37 illustrates another exemplary holding device 500. The holding device 500 is similar to that of FIGS. 34-35 and thus includes a handle 502, a shaft 504 projecting from the handle 502, and a holding fork 506 provided at a distal end of the shaft 504. However, in this embodiment, the holding fork 506 may be sized to be arranged about one of the longitudinal sections of the bone plate 28 (as opposed to the bridging sections) for more firmly holding the bone plate 28 down against the sternum 10 when inserting the fixation devices 30.

FIG. 38 illustrates yet another exemplary holding device 600. The holding device 600 is similar to that of FIG. 36 and thus includes a handle 602, a shaft 604 projecting from the handle 602, and a holding ball 610 provided at a distal end of the shaft 604. The holding ball 610 may be sized to be positioned within one of the openings 34 of the bone plate 28. The holding ball 610 may include a spike 612 for engaging and gripping underlying bone for more firmly holding the bone plate 28 down against the sternum 10 when inserting the fixation devices 30.

The sternal closure systems of this disclosure may achieve definitive sternal fixation in a manner in which both a cerclage and bone plate can be simultaneously tensioned/implanted. Various components (e.g., plate, anchor, carrier, etc.) of the system may be made of a PEEK material that can be bent to match a patient's anatomy. The bone plates and carriers of the system may also be anatomically shaped.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure. In addition, the various figures accompanying this disclosure are not necessarily to scale, and some features may be exaggerated or minimized to show certain details of a particular component or arrangement.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A sternal closure system, comprising:
a bone plate including an opening; and
a fixation device deployment assembly including a locking tab, a molly anchor, and a deployment pin,
wherein when the locking tab is engaged to the fixation device deployment assembly, the fixation device deployment assembly is configured to insert the molly anchor through the opening of the bone plate, and when the locking tab is disengaged from the fixation device deployment assembly, the fixation device deployment assembly is configured to cause the deployment pin to move the molly anchor from a relaxed position to a deployed position.

2. The sternal closure system as recited in claim 1, wherein the fixation device deployment assembly includes a pin inserter and a pusher having a shaft that is receivable within a through-bore of the pin inserter.

3. The sternal closure system as recited in claim 2, wherein the through-bore extends along a longitudinal axis of the pin inserter and extends through both a handle portion and a shaft portion of the pin inserter.

4. The sternal closure system as recited in claim 3, wherein the shaft portion of the pin inserter is accommodated within a cannulation of the molly anchor at a location between a first head portion of the molly anchor and a second head portion of the deployment pin.

5. The sternal closure system as recited in claim 4, wherein the deployment pin is preassembled to the molly anchor such that the second head portion of the deployment pin is countersunk within the cannulation relative to the first head portion.

6. The sternal closure system as recited in claim 4, wherein the second head portion of the deployment pin abuts against a circumferential ledge located within the cannulation of the molly anchor when the molly anchor is in the relaxed position.

7. The sternal closure system as recited in claim 6, wherein, when the locking tab is disengaged from the shaft of the pusher, the deployment pin is movable into engagement with the molly anchor to cause a first deployable leg and a second deployable leg of the molly anchor to splay apart and thereby transition the molly anchor from the relaxed position to the deployed position.

8. The sternal closure system as recited in claim 2, wherein the shaft extends from a proximal end section of the pusher, and further wherein the proximal end section includes first diameter that is greater than a second diameter of the shaft.

9. The sternal closure system as recited in claim 2, wherein the locking tab is configured to engage the shaft of the pusher to prevent the shaft from engaging the deployment pin.

10. The sternal closure system as recited in claim 9, wherein, when the locking tab is disengaged from the shaft of the pusher, the deployment pin is movable into engagement with the molly anchor to cause a first deployable leg and a second deployable leg of the molly anchor to splay apart and thereby transition the molly anchor from the relaxed position to the deployed position.

11. The sternal closure system as recited in claim 1, wherein the deployment pin is movably accommodated within a cannulation of the molly anchor.

12. The sternal closure system as recited in claim 1, wherein the molly anchor includes a head portion and a body portion having a first deployable leg and a second deployable leg that each extend distally from the head portion.

13. The sternal closure system as recited in claim 1, wherein the bone plate includes:
a body including a first longitudinal section, a second longitudinal section, a bridging section extending between the first longitudinal section and the second longitudinal section, a bone contacting surface, and an outer surface opposite of the bone contacting surface; and
a ridge protruding from the bone contacting surface and configured to align and center the bone plate relative to a bone.

14. The sternal closure system as recited in claim 1, wherein the bone plate includes:
a body having a first longitudinal section, a second longitudinal section, a bridging section extending between the first longitudinal section and the second longitudinal section, a bone contacting surface, and an outer surface opposite of the bone contacting surface;
a first longitudinal slot provided within the first longitudinal section; and
a second longitudinal slot provided within the second longitudinal section.

15. The sternal closure system as recited in claim 1, comprising a holding device including a holding fork or a holding ball configured to engage the bone plate for holding the bone plate down against a bone.

16. A surgical method, comprising:
positioning a bone plate relative to a sternum;
advancing a molly anchor of a fixation device deployment assembly through an opening of the bone plate and into the sternum while a locking tab is secured to the fixation device deployment assembly;
removing the locking tab from the fixation device deployment assembly; and
advancing a deployment pin to cause the molly anchor to transition from a relaxed position to a deployed position relative to the sternum.

17. The surgical method as recited in claim 16, wherein advancing the molly anchor includes:
applying a first force to a pusher of the fixation device deployment assembly with the locking tab secured to a shaft of the pusher.

18. The surgical method as recited in claim 17, wherein advancing the deployment pin includes:
applying a second force to the pusher with the locking tab removed from the shaft of the pusher.

19. The surgical method as recited in claim 18, wherein applying the second force advances the deployment pin distally into a cannulation of the molly anchor to release the molly anchor from the fixation device deployment assembly.

20. The surgical method as recited in claim 19, wherein advancement of the deployment pin distally into the cannulation causes a first deployable leg and a second deployable leg of the molly anchor to splay apart and thereby transition the molly anchor from the relaxed position to the deployed position.
